(19) United States
(12) Patent Application Publication
(10) Pub. No.: US 2014/0356885 A1
(43) Pub. Date: Dec. 4, 2014

(54) Reducing Non-Specifically Bound Molecules on Supports

(71) Applicants: Rafael Bartz, Newark, DE (US); Pratap Singh, Wilmington, DE (US); Liping Geng, Newark, DE (US); Gladys Privon, Newark, DE (US)

(72) Inventors: Rafael Bartz, Newark, DE (US); Pratap Singh, Wilmington, DE (US); Liping Geng, Newark, DE (US); Gladys Privon, Newark, DE (US)

(73) Assignee: Siemens Healthcare Diagnostics Inc., Tarrytown, NY (US)

(21) Appl. No.: 13/905,459

(22) Filed: May 30, 2013

(51) Int. Cl. G01N 33/53

(52) U.S. Cl. CPC G01N 33/5306
USPC 435/7.72; 436/501

(57) ABSTRACT

Methods and reagents are disclosed for preparing a support for reaction of the support with an assay molecule. In the method the support is treated with a detergent at a concentration of about 0.01% to about 5% (by weight) at a temperature of about 4° C. to about 50° C. for a period of about 1 hour to about 24 hours and subsequently the support is washed. The support is contacted with the assay molecule under conditions for covalently binding the assay molecule to the support to form a conjugate of the support and the assay molecule.

### BACKGROUND

The disclosure relates to the treatment of supports to prepare them for conjugation with an assay molecule so that the number of non-specifically bound molecules on the support is reduced. The disclosure also relates to methods for conjugating assay molecules to supports.

In the fields of medicine and clinical chemistry, many studies and determinations of physiologically reactive species such as cells, proteins, enzymes, cofactors, nucleic acids, substrates, antigens and antibodies, for example, are carried out using conjugates involving assay molecules such as, for example, specific binding pair (sbp) members or members of a signal-producing system (sps), e.g., labels, conjugated to supports such as, for example, particles. Various assay techniques that involve the binding of sbp members are known. These assay techniques generally also involve an sps member, e.g., a label, used in the detection part of the assay.

The assay molecule of a conjugate of a support and an assay molecule is desirably irremovably bound to the support usually by covalent binding or non-covalent binding involving molecules having strong affinity for one another. For optimal performance of the conjugates, it is important to remove substantially all non-specifically bound molecules including any assay molecules not irremovably bound to the support. Rigorous washing conditions using various wash buffers are normally employed for removal of these non-specifically bound molecules from a support prior to conjugating the support to an assay molecule. These washing conditions are often labor intensive and may become a limiting step when the conjugate reagent is produced on a manufacturing scale.

There is, therefore, a need to develop a procedure for reducing to a negligible level the amount of non-specifically bound assay molecules and other molecules on a conjugate of a support and an assay molecule.

### SUMMARY

One example of a method in accordance with the principles disclosed herein is a method of preparing a support for reaction of the support with an assay molecule. In the method the support is treated with a detergent at a concentration of about 0.01% to about 5% (by weight) at a temperature of about 4° C. to about 50° C. for a period of about 1 hour to about 24 hours and subsequently the support is washed.

Another example of a method in accordance with the principles disclosed herein is a method of preparing a conjugate of a support and an assay molecule. A support that is activated for covalently binding to an assay molecule is treated with a detergent to form a treated support, which is contacted with the assay molecule under conditions for covalently binding the assay molecule to the support to form a conjugate of the support and the assay molecule.

Another example of a method in accordance with the principles disclosed herein is a method of preparing a conjugate of a particulate latex support and a small assay molecule. A particulate latex support that is activated for covalently binding to a small assay molecule is treated with a detergent to form a treated particulate latex support, which is contacted with the small assay molecule under conditions for covalently binding the small assay molecule to the particulate latex support to form a conjugate of the particulate latex support and the small assay molecule.

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIG. 1 is a graph depicting a dose-dependent response for ouabain-coupled chemibead reagents as an example of a method in accordance with the principles described herein.

FIG. 2 is a graph depicting a dose-dependent normalized response for the method of FIG. 1. Normalized response means the response expressed as a percent of the response for zero digoxin concentration.

FIG. 3 is a graph depicting a dose-dependent response for B12-coupled chemibead reagents as an example of a method in accordance with the principles described herein.

FIG. 4 is a graph depicting a dose-dependent normalized response for the method of FIG. 3.

### DETAILED DESCRIPTION OF SPECIFIC EMBODIMENTS

### General Discussion

As indicated above, in some examples the present disclosure is directed to a method of preparing a conjugate of a support and an assay molecule. A support that is activated for covalently binding to an assay molecule is treated with a detergent to form a detergent-treated support, which then is contacted with the assay molecule under conditions for covalently binding the assay molecule to the support to form a conjugate of the support and the assay molecule. Treatment of the activated support with detergent results in a support that, subsequent to reaction with an assay molecule, is substantially free from non-specifically bound molecules, which may include non-covalently bound assay molecules as well as other non-specifically bound molecules.

The phrase “non-covalently bound assay molecules” refers to assay molecules that become attached to a support by other than a covalent bond during the reaction process wherein an assay molecule is reacted with the support to covalently bind the assay molecule to the support. Assay molecules, as well as other molecules, may become non-covalently bound to the support as the result of one or more of hydrogen bonding, van der Waals forces, electrostatic forces, hydrophobic effects, physical entrapment in the matrix of the support, and charged interactions, for example. As mentioned above, non-specifically bound assay molecules and other molecules are undesirable when the support is utilized in an assay.

The phrase “other non-specifically bound molecules” refers to molecules other than assay molecules that may become attached to a support non-specifically by, for example, one or more of hydrogen bonding, van der Waals forces, electrostatic forces, hydrophobic effects, physical entrapment in the matrix of the support, and charged interactions, for example.

The detergent that is employed to prepare the detergent-treated support may be a non-ionic detergent or an ionic detergent (anionic detergent, cationic detergent, or amphoteric detergent), for example, depending on the chemical nature of the support. The detergents that may be employed include both synthetic detergents and natural detergents. In some examples, the detergent is selected from the group consisting of polyoxyethylene detergents, polyoxypropylene detergents, and polyol detergents.

In some examples in accordance with the principles described herein, the detergent is a non-ionic detergent comprising a chain of at least 10 repeating ethylene oxide units or propylene oxide units or a combination of ethylene oxide and propylene oxide units. In some embodiments the chain is linear as opposed to branched. In some embodiments the non-ionic detergent comprises a chain of at least about 15, or at least about 20, or at least about 30, or at least about 35 repeating units as mentioned above. The number of repeating units in the chain is usually not greater than about 50, or not greater than about 45, or not greater than about 40, for example. When the chain comprises a combination of repeating ethylene oxide units and propylene oxide units, the ratio of ethylene oxide units to propylene oxide units is about 1:1, or about 2:1, or about 3:1, or about 1:2, or about 1:3, or about 1:4, or about 1:5, or about 1:6, or about 1:7, or about 1:8, for example. In the above embodiments the repeating ethylene oxide units and propylene oxide units may alternate. Each repeating ethylene oxide unit may be a segment of ethylene oxide units where the segments have the same length or different lengths, i.e., may comprise the same number of ethylene oxide units or a different number of ethylene oxide units. Each repeating propylene oxide unit may be a segment of propylene oxide units where the segments have the same length or different lengths, i.e., may comprise the same number of propylene oxide units or a different number of propylene oxide units. For example, in some embodiments the detergent comprises terminal ethylene oxide segments comprising about 1 to about 5, or about 1 to about 4, or about 1 to about 3, or about 1 to about 2, or about 2 to about 5, or about 2 to about 4, or about 2 to about 3, or about 3 to about 5, or about 4 to about 5 ethylene oxide units and an internal propylene oxide segment comprising about 10 to about 30, or about 10 to about 25, or about 10 to about 20, or about 15 to about 30, or about 15 to about 25, or about 15 to about 20, or about 20 to about 30, or about 20 to about 25 propylene oxide units.

In some embodiments the chain may comprise repeating ethylene oxide units such as, for example, —(CH₂CH₂O)ₚ— wherein p is about 15 to about 40, or about 20 to about 40, or about 25 to about 40, or about 15 to about 35, or about 20 to about 35, or about 25 to about 35, or about 30 to about 35, and so forth. In some embodiments the chain may comprise repeating propylene oxide units such as, for example, —(CH₂CH(CH₃)O)_{q}— wherein q is about 15 to about 30, or about 20 to about 30, or about 25 to about 30, or about 15 to about 25, or about 20 to about 25, or about 25 to about 35, or about 30 to about 35, and so forth.

In some embodiments the chain may comprise a combination of ethylene oxide units and propylene units such as, for example, —(CH₂CH₂O)ₛ—(CH₂CH(CH₃)O)ₜ— wherein (s+t) is at least about 10, or at least about 15, or at least about 20, or at least about 25 carbon atoms, or at least about 30, or at least about 35 and usually no more than about 40, or no more than about 35. In some embodiments (s+t) is about 15 to about 40, or about 20 to about 40, or about 25 to about 40, or about 15 to about 35, or about 20 to about 35, or about 25 to about 35, or about 30 to about 35, about 15 to about 30, or about 20 to about 30, or about 25 to about 30, or about 15 to about 25, for example. In some embodiments, as long as (s+t) is as defined above, s is about 1 to about 30, or about 1 to about 20, or about 1 to about 10, or about 1 to about 5, or about 5 to about 30, or about 5 to about 20, or about 5 to about 10, or about 10 to about 30, or about 10 to about 20, or about 10 to about 15, or about 15 to about 30, or about 15 to about 25, or about 15 to about 20, or about 20 to about 30, or about 20 to about 25, or about 25 to about 30, and t is about 5 to about 30, or about 5 to about 20, or about 5 to about 10, or about 10 to about 30, or about 10 to about 20, or about 10 to about 15, or about 15 to about 30, or about 15 to about 25, or about 15 to about 20, or about 20 to about 30, or about 20 to about 25, or about 25 to about 30.

One or more hydrogens of the chain may be substituted with alkyl of about 1 to about 5, or about 1 to about 4, or about 1 to about 3, or about 1 to about 2, or about 2 to about 5, or about 2 to about 4, or about 2 to about 3, or about 3 to about 5, or about 3 to about 4 carbon atoms, sometimes referred to herein as lower alkyl. Other substituents on the chain in place of one or more hydrogens include, for example, keto, amino, alkyl phenol, and the like. In many embodiments the number of substituents on the chain is no greater than 3, or no greater than 2 or no greater than 1. In some embodiments one terminus of the chain may be methyl, hydroxyl, or phenyl, for example. In some embodiments the other terminus of the chain may be methyl, hydroxyl, or phenyl, for example.

Particular examples of detergents comprising a combination of ethylene oxide units and propylene oxide units include the detergents PLURONIC® 25R2, PLURONIC® 25R1, PLURONIC® 25R4, PLURONIC® 31R1, PLURONIC® 31R2, PLURONIC® 17R1, PLURONIC® 17R2, PLURONIC® 10R5, PLURONIC® L123, and PLURONIC® L31, for example. Other examples of detergents comprising a combination of ethylene oxide units and propylene oxide units include the detergents DOWFAX® 63N10, DOWFAX® 63N13, DOWFAX® 63N30, DOWFAX® 63N40, DOWFAX® 20A612, DOWFAX® DF101, DOWFAX® DF111, and DOWFAX® DF112, for example.

In some examples in accordance with the principles described herein, the detergent is selected from the group consisting of polyoxyethylene alkylphenols, polyoxyethylene alcohols, polyoxyethylene esters of fatty acids, polyoxyethylene mercaptans, and polyoxyethylene alkylamides, and mixtures of two or more of the above. In some examples in accordance with the principles described herein, the detergent is selected from the group consisting of polyols such as, but not limited to pentaerythritol monolaureate, sorbitol monostearate, octyl-β-D-glucopyranoside, decaglycerol monolaureate, and decyl-β-D-maltopyranoside and mixtures of two or more of the above. In some examples, the detergent may be a mixture of two or more of a polyoxyethylene detergent, a polyoxypropylene detergent and a polyol.

Particular examples of synthetic detergents include, but are not limited to, TRITON™ X-100, TRITON™ N-101, TRITON™ X-114, TRITON™ X-405, TRITON™ SP-135, TWEEN® 20 (polyoxyethylene (20) sorbitan monolaurate), TWEEN® 80 (polyoxyethylene (20) sorbitan monooleate), DOWFAX®, ZONYL®, pentaerythrityl palmitate, ADOGEN® 464, ALKANOL® 6112 surfactant, allyl alcohol 1,2-butoxylate-block-ethoxylate HLB 6, BRIJ®, ethylenediamine tetrakis(ethoxylate-block-propoxylate) tetrol, IGEPAL®, MERPOL®, poly(ethylene glycol), 2-[ethyl[(heptadecafluorooctyl)-sulfonyl]amino]ethyl ether, polyethylene-block-poly(ethylene glycol), polyoxyethylene sorbitan tetraoleate, polyoxyethylene sorbitol hexaoleate, TERGITOL® NP-9, GAFAC® (RHODAFAC®, an alkyl polyoxyethylene glycol phosphate ester such as, for example, alpha-dodecyl-omega-hydroxypoly(oxy-1,2-ethanediyl)phosphate), and EP110®, for example. Naturally-occurring detergents that may be employed include, but are not limited to, saponins and bile salts, for example.

The amount or concentration of detergent employed to prepare the detergent-treated support depends on the nature of the detergent, the nature of the support, the nature of the assay molecule, the reaction conditions, and the nature of the signal producing system, for example. In some examples in accordance with the principles described herein, the amount of the detergent is about 0.01% to about 5%, or about 0.01% to about 4%, or about 0.01% to about 3%, or about 0.01% to about 2%, or about 0.01% to about 1%, or about 0.1% to about 5%, or about 0.1% to about 4%, or about 0.1% to about 3%, or about 0.1% to about 2%, or about 0.1% to about 1%, or about 1% to about 5%, or about 1% to about 4%, or about 1% to about 3%, or about 1% to about 2%, for example (percent is by weight).

The treatment may be carried out in a suitable medium, which may be, for example, an aqueous buffered medium. The nature of the medium is dependent on the nature of the detergent, the nature of the support, the nature of the assay molecule, and the desired performance characteristics of the resulting product, for example. In some examples the medium is an aqueous solution that comprises primarily water and may include from 0.1 to about 40 volume percent of a cosolvent such as, for example, a water miscible organic solvent, e.g., an alcohol, an ether or an amide. The aqueous solution may also comprise a buffer for pH control, for example. Some of the factors that influence the selection of a pH for the aqueous solution include, but are not limited to, the nature of the detergent, the nature of the support, the nature of the assay molecule, and the desired performance characteristics of the resulting product, for example. In some examples, the pH for the aqueous solution may be in the range of about 4 to about 11, or in the range of about 5 to about 10, or in the range of about 6.5 to about 9.5, for example.

Various buffers may be used to achieve the desired pH and maintain the pH during the method. Illustrative buffers include borate, phosphate, citrate, carbonate, TRIS, and barbital, for example. The particular buffer employed is not critical, but in an individual treatment one or another buffer or a combination of buffers may be preferred. Various ancillary materials may be employed in the above methods. For example, in addition to buffers the medium may comprise one or more stabilizers for the medium and for the reagents employed. All of the above materials are present in a concentration or amount sufficient to achieve the desired effect or function.

Other conditions for the treatment of the activated support with a detergent are those necessary to achieve the desired result of rendering the treated support substantially free from non-covalently molecules upon subsequent reaction of the detergent-treated support with an assay molecule. The temperature of the treatment of the activated support with detergent is dependent on the nature of the detergent, the nature of the support, the nature of the assay molecule, and the desired performance characteristics of the resulting product, for example. In some examples in accordance with the principles described herein, the temperature during the treatment of the activated support with detergent is about 4° C. to about 50° C., or about 4° C. to about 40° C., or about 4° C. to about 30° C., or about 10° C. to about 50° C., or about 10° C. to about 40° C., or about 10° C. to about 30° C., or about 15° C. to about 50° C., or about 15° C. to about 40° C., or about 15° C. to about 30° C., or about 15° C. to about 20° C., or about 20° C. to about 50° C., or about 20° C. to about 40° C., or about 20° C. to about 30° C., or about 25° C. to about 40° C., for example.

The duration of the treatment of the activated support with detergent is dependent on the nature of the detergent, the nature of the support, the nature of the assay molecule, the temperature of the treatment, and the desired performance characteristics of the resulting product, for example. In some examples in accordance with the principles described herein, the duration of the treatment of the activated support with detergent is about 1 hour to about 24 hours, or about 1 hour to about 20 hours, or about 1 hour to about 16 hours, or about 1 hour to about 12 hours, or about 1 hour to about 8 hours, or about 2 hours to about 24 hours, or about 2 hours to about 20 hours, or about 2 hours to about 16 hours, or about 2 hours to about 12 hours, or about 2 hours to about 8 hours, or about 4 hours to about 24 hours, or about 4 hours to about 20 hours, or about 4 hours to about 16 hours, or about 4 hours to about 12 hours, or about 4 hours to about 8 hours, for example.

The phrase “substantially free” means that the support of the assay molecule-support conjugate comprises less than about 0.1%, or less than about 0.05%, or less than about 0.01%, or less than about 0.005%, or less than about 0.001%, for example, of such non-specifically bound molecules, including non-covalently bound molecules such as, for example, non-covalently bound assay molecules and other non-specifically bound molecules.

Following treatment of the activated support with detergent, the support may be separated from the treatment medium and may also be subjected to one or more washing steps using an aqueous buffered medium. The support may be separated from the treatment medium and/or the washing medium by one or more of centrifugation, filtration, microfiltration (such as, for example, ultrafiltration, diafiltration, and gel filtration), magnetic separation, and ion exchange chromatographic separation, for example.

The support may be comprised of an organic or inorganic, solid or fluid, water insoluble material, which may be transparent or partially transparent. The support can have any of a number of shapes, such as a particle (particulate support) including a bead, a film, a membrane, a tube, a well, a strip, a rod, a fiber, and planar surfaces such as, e.g., plate, sheet and paper, for example. The support may or may not be suspendable in the medium in which it is employed. Examples of suspendable supports are polymeric materials such as latex, lipid bilayers or liposomes, oil droplets, cells and hydrogels, and magnetic particles, for example. Other support compositions include, but are not limited to, polymers, such as nitrocellulose, cellulose acetate, poly (vinyl chloride), polyacrylamide, polyacrylate, polyethylene, polypropylene, poly(4-methylbutene), polystyrene, polymethacrylate, poly(ethylene terephthalate), nylon, and poly(vinyl butyrate), for example, either used by themselves or in conjunction with other materials.

In some examples the support may be a particle. The particles have an average diameter of at least about 0.02 microns and not more than about 100 microns. In some examples, the particles have an average diameter from about 0.05 microns to about 20 microns, or from about 0.3 microns to about 10 microns. The particle may be organic or inorganic, swellable or non-swellable, porous or non-porous, preferably of a density approximating water, generally from about 0.7 g/mL to about 1.5 g/mL, and composed of material that can be transparent, partially transparent, or opaque. The particles can be biological materials such as cells and microorganisms, e.g., erythrocytes, leukocytes, lymphocytes, hybridomas, streptococcus, Staphylococcus aureus, and E. coli, viruses, for example. The particles can also be particles comprised of organic and inorganic polymers, liposomes, latex particles, magnetic or non-magnetic particles, phospholipid vesicles, chylomicrons, lipoproteins, and the like. In some examples, the particles are chromium dioxide (chrome) particles or latex particles.

Polymeric particles can be formed from addition or condensation polymers. The particles are readily dispersible in an aqueous medium. The particles can also be derived from naturally occurring materials, naturally occurring materials that are synthetically modified, and synthetic materials. Among organic polymers of particular interest are polysaccharides, particularly cross-linked polysaccharides, such a agarose, which is available as SEPHAROSE®, dextran, available as SEPHADEX® and SEPHACRYL®, cellulose, starch, and the like; addition polymers, such as polystyrene, polyvinyl alcohol, homopolymers and copolymers of derivatives of acrylate and methacrylate, particularly esters and amides having free hydroxyl functionalities, for example.

A detergent-treated support may then be used to prepare a conjugate of an assay molecule and the detergent-treated support. A reactive functionality or functional group on the support may be employed to link an assay molecule to the support whereby the assay molecule becomes covalently bound to the support. The phrase “assay molecule” refers to any molecule that is employed as part of an assay for the determination of an analyte in a sample suspected of containing such analyte. In some examples, the assay molecule is a member of a specific binding pair (sbp), or a member of a signal producing system (sps), for example. Functional groups on the assay molecule, e.g., sbp member or sps member, may be present naturally or may be introduced synthetically and are discussed more fully below.

The phrase “activated support” refers to a support that is activated for covalently binding to an assay molecule. Activation includes the presence on the surface of the support of one or more functional groups, which may be naturally occurring on the surface of the support or introduced synthetically on the surface of the support.

The nature of the functional groups employed on the activated support is dependent on one or more of the nature of the support, the nature of the functional group on an assay molecule, the nature of any coating such as, for example, a polysaccharide on the support, and flexibility in incorporation of functional groups on the support, for example. The functional groups on the support may be naturally present or may be introduced synthetically by techniques that are well known in the art. The term “functional group” refers to a functionality that can react with a corresponding reactive functionality on another molecule to form a covalent bond. Such reactive functionalities include, by way of illustration and not limitation, aldehyde, carboxy, amino, imino, sulfhydryl and hydroxy, for example. A large number of suitable functional groups are available for attaching to amino groups (amine reactive functional groups), carboxy groups (carboxy reactive functional groups), sulfhydryls (sulfhydryl reactive functional groups), and alcohols (alcohol reactive functional groups), for example. Such functional groups include, but are not limited to, activated esters including, e.g., carboxylic esters, imidic esters, sulfonic esters and phosphate esters; activated nitrites; aldehydes; ketones; maleimides; haloalkylamides; and alkylating agents, for example.

In some examples functional groups are present on the support by means of a linking group, which may comprise a chain of from 1 to about 60 or more atoms, or 1 to about 50 atoms, or 1 to about 40 atoms, or 1 to 30 atoms, or about 1 to about 20 atoms, or about 1 to about 10 atoms, or about 5 to about 60 or more atoms, or about 5 to about 50 atoms, or about 5 to about 40 atoms, or about 5 to 30 atoms, or about 5 to about 20 atoms, or about 5 to about 10 atoms, each independently selected from the group normally consisting of carbon, oxygen, sulfur, nitrogen, and phosphorous, usually carbon and oxygen. The number of heteroatoms in the linking group may range from about 0 to about 8, from about 1 to about 6, or about 2 to about 4, and include heteroatoms that may be present in a functional group on the linking group. The atoms of the linking group may be substituted with atoms other than hydrogen such as, for example, one or more of carbon, oxygen and nitrogen in the form of, e.g., alkyl, aryl, aralkyl, hydroxyl, alkoxy, aryloxy, or aralkoxy groups. As a general rule, the length of a particular linking group can be selected arbitrarily to provide for convenience of synthesis with the proviso that there is minimal interference caused by the linking group with the ability of the linked molecules to perform their particular function such as, for example, their function in an assay. The linking group may be aliphatic or aromatic. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers, ureas, thioureas, guanidines, azo groups, thioethers, carboxylate and so forth. The linking group may also be a macro-molecule such as polysaccharides, peptides, proteins, nucleotides, and dendrimers. In some examples, the linking group is ouabain or vitamin B12.

In one particular example, the support may comprise one or more polysaccharide derivative coatings that are bound either covalently or non-covalently to a surface of the support. The polysaccharide may comprise aldehyde groups, which can react with a corresponding functional group of the assay molecule. For example, the reaction between an aldehyde group and an sbp member may be by means of, for example, Schiff's base formation between an alkyl amine or an aryl amine of the assay molecule and the aldehyde group of the polysaccharide that is bound to the support. The reaction may be by means of reductive amination involving the aldehyde group and an amine group of the assay molecule. In one example, the aldehyde functionality may react with a corresponding amine group on the assay molecule whereby the assay molecule and the support become covalently bound.

In the context of the present disclosure, the term “polysaccharide” refers to a macromolecule that is a polymeric carbohydrate in forms that are both miscible (soluble) and immiscible (insoluble in) with water, which includes, for example, dextran (available, e.g., as a powder (water soluble) or as SEPHADEX® and SEPHAROSE®), dextran derivatives, agarose (available, e.g., as SEPHAROSE®), cellulose, starch, glycogen and chitin and including derivatives of the above. The term “derivative” means that the polysaccharide is functionalized with a functional group for reaction with a functional group of another entity such as, for example, a support. In some instances the polysaccharide is dextran or a dextran derivative. The polysaccharide derivative comprises a functional group such as an amine group, an amine reactive functional group or an alcohol reactive functional group, examples of which are set forth below. In some examples the functional groups of the support are amine reactive functional groups and the functional groups of the polysaccharide are amine functional groups. In some examples the functional groups of the support are amine groups and the functional groups of the polysaccharide are amine reactive functional groups.

The nature of the functional groups employed on an assay molecule is dependent on one or more of the nature of the functional groups of the activated support, the nature of the assay molecule, and flexibility of incorporating functional groups on the assay molecule, for example. The functional groups on the support and the functional groups on the assay molecule including any functional groups on a linking group if employed should be reactive with one another. The functional groups on the assay molecule may be naturally present or may be introduced synthetically by techniques that are well known in the art. Such reactive functionalities are as described above for the functionalities on an activated support and include, by way of illustration and not limitation, aldehyde, carboxy, amino, imino, sulfhydryl and hydroxy, for example. A large number of suitable functional groups are available for attaching to amino groups (amine reactive functional groups), carboxy groups (carboxy reactive functional groups), sulfhydryls (sulfhydryl reactive functional groups), and alcohols (alcohol reactive functional groups), for example. Such functional groups include, but are not limited to, activated esters including, e.g., carboxylic esters, imidic esters, sulfonic esters and phosphate esters; activated nitrites; aldehydes; ketones; maleimides; haloalkylamides; and alkylating agents, for example.

Uses of Supports with Bound Assay Molecules

As mentioned above, a support with bound assay molecule may be employed in an assay for determining one or both of the presence and amount of an analyte in a sample. A sample to be analyzed is obtained from a sample source. The sample to be tested may be non-biological or biological. “Non-biological samples” are those that do not relate to a biological material and include, for example, soil samples, water samples, air samples, samples of other gases and mineral samples. The phrase “biological sample” refers to any biological material such as, for example, body fluid, body tissue, body compounds and culture media. The sample may be a solid, semi-solid or a fluid (a liquid or a gas) from any source.

In some examples the sample may be a body excretion, a body aspirant, a body excisant or a body extractant. The body is usually that of a mammal and in some embodiments the body is a human body. Body excretions are those substances that are excreted from a body (although they also may be obtained by excision or extraction) such as, for example, urine, feces, stool, vaginal mucus, semen, tears, breath, sweat, blister fluid and inflammatory exudates. Body excisants are those materials that are excised from a body such as, for example, skin, hair and tissue samples including biopsies from organs and other body parts. Body aspirants are those materials that are aspirated from a body such as, for example, mucus, saliva and sputum. Body extractants are those materials that are extracted from a body such as, for example, whole blood, plasma, serum, spinal fluid, cerebral spinal fluid, lymphatic fluid, synovial fluid and peritoneal fluid.

The analyte is a substance of interest or the compound or composition to be detected and/or quantitated. Analytes include, by way of illustration and not limitation, therapeutic drugs, drugs of abuse, metabolites, pesticides, volatile organic compounds, semi-volatile organic compounds, non-volatile organic compounds, proteins, polysaccharides, pollutants, toxins, lipids and nucleic acids, (DNA, RNA), for example. The analyte is a substance of interest or the compound or composition to be detected and/or quantitated. Analytes include, for example, drugs, metabolites, pesticides and pollutants. Representative analytes, by way of illustration and not limitation, include alkaloids, steroids, lactams, aminoalkylbenzenes, benzheterocyclics, purines, drugs derived from marijuana, hormones, polypeptides which includes proteins, immunosuppressants, vitamins, prostaglandins, tricyclic antidepressants, anti-neoplastics, nucleosides and nucleotides including polynucleosides and polynucleotides, miscellaneous individual drugs which include methadone, meprobamate, serotonin, meperidine, lidocaine, procainamide, acetylprocainamide, propranolol, griseofulvin, valproic acid, butyrophenones, antihistamines, chloramphenicol, anticholinergic drugs, and metabolites and derivatives of all of the above. Also included are metabolites related to disease states, aminoglycosides, such as gentamicin, kanamicin, tobramycin, and amikacin, and pesticides such as, for example, polyhalogenated biphenyls, phosphate esters, thiophosphates, carbamates and polyhalogenated sulfenamides and their metabolites and derivatives. The term analyte also includes combinations of two or more of polypeptides and proteins, polysaccharides and nucleic acids. Such combinations include, for example, components of bacteria, viruses, chromosomes, genes, mitochondria, nuclei and cell membranes. Protein analytes include, for example, immunoglobulins, cytokines, enzymes, hormones, cancer antigens, nutritional markers and tissue specific antigens. Such proteins include, by way of illustration and not limitation, protamines, histones, albumins, globulins, scleroproteins, phosphoproteins, mucoproteins, chromoproteins, lipoproteins, nucleoproteins, glycoproteins, T-cell receptors, proteoglycans, HLA, unclassified proteins, e.g., somatotropin, prolactin, insulin, pepsin, proteins found in human plasma, blood clotting factors, protein hormones such as, e.g., follicle-stimulating hormone, luteinizing hormone, luteotropin, prolactin, chorionic gonadotropin, tissue hormones, cytokines, cancer antigens such as, e.g., PSA, CEA, α-fetoprotein, acid phosphatase, CA19.9, CA15.3 and CA125, tissue specific antigens, such as, e.g., alkaline phosphatase, myoglobin, CPK-MB and calcitonin, and peptide hormones. Other polymeric materials of interest are mucopolysaccharides and polysaccharides. As indicated above, the term analyte further includes oligonucleotide and polynucleotide analytes such as m-RNA, r-RNA, t-RNA, DNA and DNA-RNA duplexes, for example.

Representative drug analytes, by way of illustration and not limitation, include alkaloids, steroids, lactams, aminoalkylbenzenes, benzheterocyclics, purines, drugs derived from marijuana, hormones, polypeptides which includes proteins, immunosuppressants, vitamins, prostaglandins, tricyclic antidepressants, anti-neoplastics, nucleosides and nucleotides including polynucleosides and polynucleotides, miscellaneous individual drugs which include methadone, meprobamate, serotonin, meperidine, lidocaine, procainamide, acetylprocainamide, propranolol, griseofulvin, valproic acid, butyrophenones, antihistamines, chloramphenicol, anticholinergic drugs, and metabolites and derivatives of all of the above.

Also included within the term analyte are metabolites related to disease states, aminoglycosides, such as gentamicin, kanamicin, tobramycin, and amikacin, and pesticides such as, for example, polyhalogenated biphenyls, phosphate esters, thiophosphates, carbamates and polyhalogenated sulfenamides and their metabolites and derivatives.

The term analyte also includes combinations of two or more of polypeptides and proteins, polysaccharides and nucleic acids. Such combinations include, for example, components of bacteria, viruses, chromosomes, genes, mitochondria, nuclei and cell membranes. Protein analytes include, for example, immunoglobulins, cytokines, enzymes, hormones, cancer antigens, nutritional markers and tissue specific antigens. Such proteins include, by way of illustration and not limitation, protamines, histones, albumins, globulins, scleroproteins, phosphoproteins, mucoproteins, chromoproteins, lipoproteins, nucleoproteins, glycoproteins, T-cell receptors, proteoglycans, HLA, unclassified proteins, e.g., somatotropin, prolactin, insulin, pepsin, proteins found in human plasma, blood clotting factors, protein hormones such as, e.g., follicle-stimulating hormone, luteinizing hormone, luteotropin, prolactin, chorionic gonadotropin, tissue hormones, cytokines, cancer antigens such as, e.g., PSA, CEA, a-fetoprotein, acid phosphatase, CA19.9 and CA125, tissue specific antigens, such as, e.g., alkaline phosphatase, myoglobin, CPK-MB and calcitonin, and peptide hormones. Other polymeric materials of interest are mucopolysaccharides and polysaccharides. As indicated above, the term analyte further includes oligonucleotide and polynucleotide analytes such as m-RNA, r-RNA, t-RNA, DNA and DNA-RNA duplexes, for example.

The sample can be prepared in any convenient medium that does not interfere with an assay; an aqueous medium generally is employed. The assay comprises adding reagents for determining the presence or concentration of the analyte in the sample to a medium comprising the sample. In some examples the assay is an immunoassay and the reagents comprise at least one antibody for the analyte. An amount of a complex comprising the antibody for the analyte is measured. The phrase “complex comprising the antibody for the analyte” refers to a complex wherein the antibody for the analyte is complexed to one or more substances that may be one or more of the analyte and other substances in a sample that bind to the antibody for the analyte.

Any suitable assay may be employed for determining the analyte as long as such assay utilizes a support in the determination. The assays are conducted by combining the sample with reagents for determining the amount of the analyte in the sample. The nature of the reagents is dependent on the particular type of assay to be performed. The assay may be an immunoassay or a non-immunoassay. Various assay methods are discussed below by way of illustration and not limitation.

### General Description of Assays for an Analyte

The following discussion is by way of illustration and not limitation. The supports produced in accordance with the principles described herein may be employed in any assay that employs a support reagent. In many examples the reagents comprise at least one antibody for the analyte and the assay is generally referred to as an immunoassay as distinguished from assays that do not utilize an antibody, which are referred to as non-immunoassays. By the phrase “antibody for the analyte” is meant an antibody that binds specifically to the analyte (and in some instances to closely related structural analogs of the analyte such as metabolites of the analyte) and does not bind to any significant degree to other substances that would distort the analysis for the analyte.

Immunoassays may involve labeled or non-labeled reagents. Immunoassays involving non-labeled reagents usually comprise the formation of relatively large complexes involving one or more antibodies. Such assays include, for example, immunoprecipitin and agglutination methods and corresponding light scattering techniques such as, e.g., nephelometry and turbidimetry, for the detection of antibody complexes. Labeled immunoassays include chemiluminescence immunoassays, enzyme immunoassays, fluorescence polarization immunoassays, radioimmunoassay, inhibition assay, induced luminescence, fluorescent oxygen channeling assay, and so forth.

One general group of immunoassays that may be employed includes immunoassays using a limited concentration of antibody. Another group of immunoassays involves the use of an excess of one or more of the principal reagents such as, for example, an excess of an antibody for the analyte. Another group of immunoassays are separation-free homogeneous assays in which the labeled reagents modulate the label signal upon analyte-antibody binding reactions. Another group of assays includes labeled antibody reagent limited competitive assays for analyte that avoid the use of problematic labeled haptens. In this type of assay, a solid phase immobilized analyte is present in a constant, limited amount. The partition of a label between the immobilized analyte and free analyte depends on the concentration of analyte in the sample.

Antibodies specific for an analyte for use in immunoassays can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies.

Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

As discussed above, an antibody selected for use in an immunoassay for an analyte, for example, should specifically and preferentially bind the analyte (and its pharmaceutically active metabolites, if necessary or desired) over other ligands such as other metabolites or related substances.

Other reagents are included in the assay medium depending on the nature of the assay to be conducted. Such assays usually involve reactions between binding partners such as an analyte and a corresponding antibody or the binding between an antibody and a corresponding binding partner such as a second antibody that binds to the first antibody. Accordingly, the binding partner may be a protein, which may be an antibody or an antigen. The binding partner may be an sbp member, which is one of two different molecules, having an area on the surface or in a cavity, which specifically binds to and is thereby defined as complementary with a particular spatial and polar organization of the other molecule. The sbp members in many instances are members of an immunological pair such as antigen-antibody, although other specific binding pairs such as, for example, biotin-avidin, hormones-hormone receptors, enzyme-substrate, nucleic acid duplexes, IgG-protein A, and polynucleotide pairs such as DNA-DNA, DNA-RNA, are not immunological pairs but are included within the scope of sbp member.

Accordingly, specific binding involves the specific recognition of one of two different molecules for the other compared to substantially less recognition of other molecules. On the other hand, non-specific binding involves non-covalent binding between molecules that is relatively independent of specific surface structures. Non-specific binding may result from several factors including hydrophobic interactions between molecules. In many embodiments of assays, preferred binding partners are antibodies and the assays are referred to as immunoassays.

The immunoassays may involve labeled reagents and such assays include, for example, enzyme immunoassays, fluorescence polarization immunoassays, radioimmunoassay, inhibition assay, induced luminescence, fluorescent oxygen channeling assay, and so forth.

The assays can be performed either without separation (homogeneous) or with separation (heterogeneous) of any of the assay components or products. Homogeneous immunoassays are exemplified by the EMIT® assay (Siemens Healthcare Diagnostics Inc., Newark Del.) disclosed in Rubenstein, et al., U.S. Pat. No. 3,817,837, column 3, line 6 to column 6, line 64; immunofluorescence methods such as those disclosed in Ullman, et al., U.S. Pat. No. 3,996,345, column 17, line 59, to column 23, line 25; enzyme channeling immunoassays (“ECIA”) such as those disclosed in Maggio, et al., U.S. Pat. No. 4,233,402, column 6, line 25 to column 9, line 63; the fluorescence polarization immunoassay (“FPIA”) as disclosed, for example, in, among others, U.S. Pat. No. 5,354,693; and so forth.

In a homogeneous assay, after all of the reagents have been combined, the signal is determined and related to the amount of analyte in the sample. For example, in an EMIT® assay for an analyte, a sample suspected of containing the analyte is combined in an aqueous medium either simultaneously or sequentially with an enzyme conjugate of the analyte, i.e., an analog of the analyte, and antibody capable of recognizing the analyte. Generally, a substrate for the enzyme is added, which results in the formation of a chromogenic or fluorogenic product upon enzyme catalyzed reaction. Preferred enzymes are glucose-6-phosphate dehydrogenase and alkaline phosphatase but other enzymes may be employed. The analyte (and/or other substances in the sample that might bind to the antibody) and the moieties of the enzyme conjugate compete for binding sites on the antibody. The enzyme activity in the medium is then measured, usually by spectrophotometric means. Calibrators may also be tested in a manner similar to the testing of the sample suspected of containing the analyte. The calibrators typically contain differing, but known, concentrations of the analyte to be determined. Preferably, the concentration ranges present in the calibrators span the range of suspected analyte concentrations in unknown samples.

The aforementioned assays may be carried out using mutant glucose-6-phosphate dehydrogenase as the enzyme of the enzyme conjugate. This mutant enzyme is described in U.S. Pat. Nos. 6,090,567 and 6,033,890, the relevant disclosures of which are incorporated herein by reference. Furthermore, the assay may be conducted using antibodies for the analyte and using procedures as disclosed in U.S. Pat. Nos. 5,328,828 and 5,135,863, the relevant disclosures of which are incorporated herein by reference.

Other enzyme immunoassays are the radial partition immunoassays (RPIA) described by Giegel, et al., Clin. Chem. (1982) 28: 1894; the enzyme modulate mediated immunoassay (“EMMIA”) discussed by Ngo and Lenhoff, FEBS Lett. (1980) 116:285-288; the substrate labeled fluorescence immunoassay (“SLFIA”) disclosed by Oellerich, J. Clin. Chem. Clin. Biochem. (1984) 22:895-904; the combined enzyme donor immunoassays (“CEDIA”) disclosed by Khanna, et al., Clin. Chem. Acta (1989) 185:231-240; homogeneous particle labeled immunoassays such as particle enhanced turbidimetric inhibition immunoassays (“PETINIA”), particle enhanced turbidimetric immunoassay (“PETIA”), etc.; and the like.

Other assays include the sol particle immunoassay (“SPIA”), the disperse dye immunoassay (“DIA”); the metalloimmunoassay (“MIA”); the enzyme membrane immunoassays (“EMIA”); luminoimmunoassays (“LIA”); acridinium ester label immunoassays using paramagnetic particles as a solid phase (ADVIA Centaur immunoassays); and so forth. Other types of assays include immunosensor assays involving the monitoring of the changes in the optical, acoustic and electrical properties of an antibody-immobilized surface upon the binding of a drug. Such assays include, for example, optical immunosensor assays, acoustic immunosensor assays, semiconductor immunosensor assays, electrochemical transducer immunosensor assays, potentiometric immunosensor assays, amperometric electrode assays, and the like.

In many of the assays discussed herein for determination of an analyte, a label is employed; the label is usually part of a signal producing system (sps). The nature of the label is dependent on the particular assay format. A signal producing system may include one or more components, at least one component being a detectable label, which generates a detectable signal that relates to the amount of one or both of bound and unbound label, i.e. the amount of label bound or not bound to the analyte being detected or to an agent that reflects the amount of the analyte to be detected. The label is any molecule that produces or can be induced to produce a signal, and may be, for example, a fluorescer, radiolabel, enzyme, chemiluminescer or photosensitizer. Thus, the signal is detected and/or measured by detecting enzyme activity, luminescence, light absorbance or radioactivity, for example, as the case may be.

Suitable labels include, by way of illustration and not limitation, enzymes such as alkaline phosphatase, glucose-6-phosphate dehydrogenase (“G6PDH”) and horseradish peroxidase; ribozyme; a substrate for a replicase such as QB replicase; promoters; dyes; fluorescers, such as fluorescein isothiocyanate, rhodamine compounds, phycoerythrin, phycocyanin, allophycocyanin, o-phthaldehyde, and fluorescamine; complexes such as those prepared from CdSe and ZnS present in semiconductor nanocrystals known as Quantum dots; chemiluminescers such as isoluminol and acridinium esters, for example; sensitizers; coenzymes; enzyme substrates; radiolabels such as ¹²⁵I, ¹³¹I, ¹⁴C, ³H, ⁵⁷Co and ⁷⁵Se; particles such as latex particles, carbon particles, metal particles including magnetic particles, e.g., chromium dioxide (CrO₂) particles, and the like; metal sol; crystallite; liposomes; cells, etc., which may be further labeled with a dye, catalyst or other detectable group. Suitable enzymes and coenzymes are disclosed in Litman, et al., U.S. Pat. No. 4,275,149, columns 19-28, and Boguslaski, et al., U.S. Pat. No. 4,318,980, columns 10-14; suitable fluorescers and chemiluminescers are disclosed in Litman, et al., U.S. Pat. No. 4,275,149, at columns 30 and 31; which are incorporated herein by reference.

The label can directly produce a signal and, therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a second wavelength. Other labels that directly produce a signal include radioactive isotopes and dyes.

Alternately, the label may need other components to produce a signal, and the signal producing system would then include all the components required to produce a measurable signal. Such other components may include substrates, coenzymes, enhancers, additional enzymes, substances that react with enzymic products, catalysts, activators, cofactors, inhibitors, scavengers, metal ions, and a specific binding substance required for binding of signal generating substances.

The sps member is associated with the support. The manner of association of the sps member with the support depends on one or more of the nature of the support, the nature of the sps member, the surface area and porosity of the support and the nature of any solvent employed, for example. The association may be by adsorption of the sps member by the support, covalent bonding of the sps member to the support, for example, by covalent attachment to a functional group of the polysaccharide on the support, dissolution or dispersion of the sps member in the support, non-covalent bonding of the sps member to the support by means of binding pair members (e.g., avidin-biotin and digoxin-antibody for digoxin), for example. In this manner the sps member is “associated with” the solid support.

As used herein, the phrase “associated with” includes covalent binding of one molecule to another molecule either by a direct bond or through a spacer group, non-covalent binding of one molecule to another molecule either directly or by means of specific binding pair members bound to the moieties, incorporation of one molecule into another molecule such as by dissolving one molecule in another molecule or by synthesis, and coating one molecule on another molecule, for example. Association of an sps member such as, for example, a sensitizer or a chemiluminescent compound, with latex particles may involve incorporation during formation of the particles by polymerization, or incorporation into preformed particles, e.g., by non-covalent dissolution into the particles, for example.

As mentioned above, depending on the type of assay format, the label or other sps members can be bound to the support. In some examples, one or both of the label and sps member may be bound to an sbp member or another molecule. For example, the label can be bound covalently to an sbp member such as, for example, an antibody, a receptor for an antibody, a receptor that is capable of binding to a small molecule conjugated to an antibody, or a ligand (analyte) analog. Bonding of the label to the sbp member may be accomplished by chemical reactions that result in replacing a hydrogen atom of the label with a bond to the sbp member or may include a linking group between the label and the sbp member. Other sps members may also be bound covalently to sbp members. For example, two sps members such as a fluorescer and quencher can each be bound to a different antibody that forms a specific complex with the analyte. Formation of the complex brings the fluorescer and quencher in close proximity, thus permitting the quencher to interact with the fluorescer to produce a signal. Methods of conjugation are well known in the art. See, for example, Rubenstein, et al., U.S. Pat. No. 3,817,837, incorporated herein by reference.

Enzymes of particular interest as label proteins are redox enzymes, particularly dehydrogenases such as glucose-6-phosphate dehydrogenase, lactate dehydrogenase, etc., and enzymes that involve the production of hydrogen peroxide and the use of the hydrogen peroxide to oxidize a dye precursor to a dye. Particular combinations include saccharide oxidases, e.g., glucose and galactose oxidase, or heterocyclic oxidases, such as uricase and xanthine oxidase, coupled with an enzyme which employs the hydrogen peroxide to oxidize a dye precursor, that is, a peroxidase such as horse radish peroxidase, lactoperoxidase, or microperoxidase. Additional enzyme combinations are known in the art. When a single enzyme is used as a label, other enzymes may find use such as hydrolases, transferases, and oxidoreductases, preferably hydrolases such as alkaline phosphatase and beta-galactosidase. Alternatively, luciferases may be used such as firefly luciferase and bacterial luciferase.

Illustrative co-enzymes that find use include NAD[H], NADP[H], pyridoxal phosphate, FAD[H], FMN[H], etc., usually coenzymes involving cycling reactions. See, for example, U.S. Pat. No. 4,318,980, the disclosure of which is incorporated herein by reference.

The term “non-poly(amino acid) labels” includes those labels that are not proteins (e.g., enzymes). The non-poly(amino acid) label is capable of being detected directly or is detectable through a specific binding reaction that produces a detectable signal. The non-poly(amino acid) labels include, for example, radioisotopes, luminescent compounds, supports, e.g., particles, plates, beads, etc., polynucleotides, and the like. More particularly, the non-poly(amino acid) label can be isotopic or non-isotopic, usually non-isotopic, and can be a polynucleotide coding for a catalyst, promoter, dye, coenzyme, enzyme substrate, radioactive group, a small organic molecule (including, e.g., biotin, fluorescent molecules, chemiluminescent molecules, and the like), amplifiable polynucleotide sequence, a support such as, for example, a particle such as latex or carbon particle or chromium dioxide (chrome) particle or the like, metal sol, crystallite, liposome, cell, etc., which may or may not be further labeled with a dye, catalyst or other detectable group, and the like.

In a typical competitive heterogeneous assay, an example of a support treated in accordance with the principles described herein and further comprising an sbp member that binds to an analyte is contacted with a medium containing the sample suspected of containing the analyte and the analyte conjugated to a label that is reactive with the sps member of the present composition or with a product of the activation of the sps member. Activation of the sps member on the support produces a signal from the label if the analyte is present, which is determined by conventional techniques and is related to the amount of the analyte in the sample.

In a typical non-competitive sandwich assay, an immune sandwich complex is formed in an assay medium. The complex comprises the analyte, an sbp member (first sbp member) covalently attached to a support treated in accordance with the principles described herein, and a second sbp member that binds to the analyte or to the first sbp member. Subsequently, the immune sandwich complex is detected and is related to the amount of analyte in the sample. The immune sandwich complex is detected by virtue of the presence in the complex of one or more of a label of the support and a label of the second sbp member.

Some known assays utilize a signal producing system that employs first and second sps members. The sps members may be related in that activation of one member of the sps produces a product such as, e.g., light, which results in activation of another member of the sps. In one approach in a sandwich assay, a first incubation is carried out using a support with an sbp member for the analyte attached thereto, which is contacted with a medium containing a sample suspected of containing the analyte. After a wash and separation step, the support of the present composition is contacted with a medium containing a second sbp member such as, for example, an antibody for the analyte, which contains a label such as an enzyme, for a second incubation period. The labels are related in that activation of one of the labels activates the other label if the analyte is present in the medium. The support is again washed and separated from the medium and either the medium or the support is examined for the presence of a signal. The presence and amount of signal is related to the presence or amount of the analyte.

In a variation of the above sandwich assay, the sample suspected of containing the analyte in a suitable medium is contacted with labeled antibody for the analyte and incubated for a period of time. Then, the medium is contacted with a support treated in accordance with the principles described herein, which comprises a label that is related to the label of the labeled antibody as discussed above. After an incubation period, the support is separated from the medium and washed to remove unbound reagents. The support or the medium is examined for the presence of a signal, which is related to the presence or amount of analyte. In another variation of the above, the sample, the support and the labeled antibody are combined in a medium and incubated in a single incubation step. Separation, wash steps and examination for signal are as described above.

In some embodiments of known assays, the sps members comprise a sensitizer such as, for example, a photosensitizer, and a chemiluminescent composition where activation of the sensitizer results in a product that activates the chemiluminescent composition. The second sps member usually generates a detectable signal that relates to the amount of bound and/or unbound sps member, i.e. the amount of sps member bound or not bound to the analyte being detected or to an agent that reflects the amount of the analyte to be detected. In accordance with the principles described herein, the support may comprise one of either the sensitizer reagent or the chemiluminescent reagent.

In one embodiment the assay is an induced luminescence immunoassay, which is described in U.S. Pat. No. 5,340,716 (Ullman, et al.) entitled “Assay Method Utilizing Photoactivated Chemiluminescent Label” (“induced luminescence assay”), which disclosure is incorporated herein by reference. In one approach the assay uses a particle incorporating a photosensitizer and a label particle incorporating a chemiluminescent compound. One or both of the particles may be coated with a polysaccharide where the particles were prepared in accordance with the principles disclosed herein. The label particle may be conjugated to an sbp member, for example, an antibody for the analyte that is capable of binding to the analyte to form a complex, or to a second sbp member to form a complex, in relation to the amount of the analyte. If the analyte is present, the photosensitizer and the chemiluminescent compound come into close proximity. The photosensitizer generates singlet oxygen and activates the chemiluminescent compound when the two labels are in close proximity. The activated chemiluminescent compound subsequently produces light. The amount of light produced is related to the amount of the complex formed, which comprises antibody for the analyte.

By way of further illustration, chemiluminescent particles may be employed, which comprise the chemiluminescent compound associated therewith such as by incorporation therein or attachment thereto. An sbp member that binds to the analyte, such as, for example, an antibody for analyte, is bound to a polysaccharide coating the particles, which are prepared in accordance with the principles disclosed herein. A second sbp member that binds to the analyte is part of a biotin conjugate. Streptavidin is conjugated to a second set of particles having a photosensitizer associated therewith. The binding of the streptavidin to this second set of particles (photosensitizer particles) may or may not involve a polysaccharide on the particles. The chemiluminescent particles are mixed with the respective portion of the sample suspected of containing an analyte and with the photosensitizer particles. With regard to the first portion of the sample, the reaction medium is incubated to allow the particles to bind to substances or components in the sample other than analyte. With regard to the second portion of the sample, the reaction medium is incubated to allow the particles to bind to the analyte by virtue of the binding of the sbp members to the analyte. Then, the medium is irradiated with light to excite the photosensitizer, which is capable in its excited state of activating oxygen to a singlet state. Because the chemiluminescent compound of one of the sets of particles is now in close proximity to the photosensitizer by virtue of the presence of the substances and/or the analyte, it is activated by singlet oxygen and emits luminescence. The medium is then examined for the amount of luminescence or light emitted, the presence thereof being related to the amount of the substances that bind to antibody for the analyte or the amount of analyte.

Another particular example of an assay that may be employed for the determination of an analyte is discussed in U.S. Pat. No. 5,616,719 (Davalian, et al.), which describes fluorescent oxygen channeling immunoassays.

The assays discussed above are normally carried out in an aqueous buffered medium at a moderate pH, generally that which provides optimum assay sensitivity. The pH for the assay medium will usually be in the range of about 4 to about 11, or in the range of about 5 to about 10, or in the range of about 6.5 to about 9.5. The pH will usually be a compromise among one or more of optimum binding of the binding members of any specific binding pairs and the pH optimum for other reagents of the assay such as members of a signal producing system, for example.

Various buffers may be used to achieve the desired pH and maintain the pH during the determination. Illustrative buffers include borate, phosphate, carbonate, tris, barbital and the like. The particular buffer employed is not critical, but in an individual assay one or another buffer may be preferred. Various ancillary materials may be employed in the above methods. For example, in addition to buffers the medium may comprise stabilizers for the medium and for the reagents employed. In some embodiments, in addition to these additives, proteins may be included, such as albumins; quaternary ammonium salts; polyanions such as dextran sulfate; binding enhancers, or the like. All of the above materials are present in a concentration or amount sufficient to achieve the desired effect or function.

One or more incubation periods may be applied to the medium at one or more intervals including any intervals between additions of various reagents mentioned above. The medium is usually incubated at a temperature and for a time sufficient for binding of various components of the reagents to occur. Moderate temperatures are normally employed for carrying out the method and usually constant temperature, preferably, room temperature, during the period of the measurement. Incubation temperatures normally range from about 5° to about 99° C. or from about 15° C. to about 70° C., or about 20° C. to about 45° C., for example. The time period for the incubation is about 0.2 seconds to about 24 hours, or about 1 second to about 6 hours, or about 2 seconds to about 1 hour, or about 1 minute to about 15 minutes, for example. The time period depends on the temperature of the medium and the rate of binding of the various reagents. Temperatures during measurements will generally range from about 10 to about 50° C. or from about 15 to about 40° C.

The concentration of analyte that may be assayed generally varies from about 10⁻⁵ to about 10⁻¹⁷ M, or from about 10⁻⁶ to about 10⁻¹⁴ M. Considerations, such as whether the assay is qualitative, semi-quantitative or quantitative (relative to the amount of erythrocytophilic drug analyte present in the sample), the particular detection technique and the concentration of the analyte normally determine the concentrations of the various reagents.

The concentrations of the various reagents in the assay medium will generally be determined by one or more of the concentration range of interest of the analyte, the nature of the assay, the antibody affinity and avidity and antibody fragmentation, for example. However, the final concentration of each of the reagents is normally determined empirically to optimize the sensitivity of the assay over the range. That is, a variation in concentration of analyte that is of significance should provide an accurately measurable signal difference. Considerations such as the nature of a signal producing system and the nature of the analyte normally determine the concentrations of the various reagents.

While the order of addition may be varied widely, there will be certain preferences depending on the nature of the assay. The simplest order of addition is to add all the materials simultaneously and determine the effect that the assay medium has on the signal as in a homogeneous assay. Alternatively, the reagents can be combined sequentially. Optionally, an incubation step may be involved subsequent to each addition as discussed above.

### Examination Step

In a next step of an assay method, the medium is examined for the presence of a complex comprising the analyte. One or both of the presence and amount of the complex indicates one or both of the presence and amount of the analyte in the sample. The phrase “measuring the amount of an analyte” refers to the quantitative, semi-quantitative and qualitative determination of the analyte. Methods that are quantitative, semi-quantitative and qualitative, as well as all other methods for determining the analyte, are considered to be methods of measuring the amount of the analyte. For example, a method, which merely detects the presence or absence of the analyte in a sample suspected of containing the analyte, is considered to be included within the scope of the examination step. The terms “detecting” and “determining,” as well as other common synonyms for measuring, are contemplated within the scope of the present embodiments.

In many instances the examination of the medium involves detection of a signal from the medium. The amount of the signal is related to the amount of the analyte in the sample. The particular mode of detection depends on the nature of the signal producing system. As discussed herein, there are numerous methods by which a label of a signal producing system can produce a signal detectable by external means, desirably by visual examination, and include, for example, electromagnetic radiation, electrochemistry, heat, radioactivity detection, chemical reagents and so forth.

Activation of a signal producing system depends on the nature of the sps members. For those sps members that are activated with light, the member is irradiated with light. For sps members that are on the surface of a particle, for example, addition of a base may result in activation. Other activation methods will be suggested to those skilled in the art in view of the disclosures herein. For some signal producing systems, no agent for activation is necessary such as those systems that involve a label that is a radioactive label, an enzyme, and so forth. For enzyme systems, addition of a substrate and/or a cofactor may be necessary.

The examination for amount of the signal also includes the detection of the signal, which is generally merely a step in which the signal is read. Luminescence or light produced from any label can be measured visually, photographically, actinometrically, spectrophotometrically or by any other convenient means to determine the amount thereof, which is related to the amount of analyte in the medium. In some instances, the signal is read using an instrument, the nature of which depends on the nature of the signal. The instrument may be a spectrophotometer, fluorometer, absorption spectrometer, luminometer, chemiluminometer, actinometer, scintillation counter, or a photographic instrument, for example. The amount of signal detected is related to the amount of the analyte present in a sample. Temperatures during measurements generally range from about 10° to about 70° C. or from about 20° to about 45° C., or about 20° to about 25° C., for example. In one approach standard curves are formed using known concentrations of the analytes to be screened. As discussed herein, calibrators and other controls may also be used.

### Kits for Conducting Assays

The reagents for conducting a particular assay may be present in a kit useful for conveniently performing an assay for the determination of an analyte. In one example, a kit comprises in packaged combination reagents for conducting an assay for the analyte, which may include an antibody for an analyte and other reagents for performing an assay, the nature of which depend upon the particular assay format and further include support reagents in accordance with the principles described herein. The reagents may each be in separate containers or various reagents can be combined in one or more containers depending on the cross-reactivity and stability of the reagents. The kit can further include other separately packaged reagents for conducting an assay such as additional sbp members, ancillary reagents such as an ancillary enzyme substrate, and so forth.

The relative amounts of the various reagents in the kits can be varied widely to provide for concentrations of the reagents that substantially optimize the reactions that need to occur during the present method and further to optimize substantially the sensitivity of the assay. Under appropriate circumstances one or more of the reagents in the kit can be provided as a dry powder, usually lyophilized, including excipients, which on dissolution will provide for a reagent solution having the appropriate concentrations for performing a method or assay. The kit can further include a written description of a method in accordance with the present embodiments as described above.

### DEFINITIONS

The following definitions are provided for terms and phrases not otherwise specifically defined above.

The phrase “at least” as used herein means that the number of specified items may be equal to or greater than the number recited.

The phrase “about” as used herein means that the number recited may differ by plus or minus 10%; for example, “about 5” means a range of 4.5 to 5.5.

The designations “first” and “second” are used solely for the purpose of differentiating between two items such as, for example, “first sps member” and “second sps member,” and are not meant to imply any sequence or order or importance to one item over another or any order of addition, for example.

The following examples further describe the specific examples of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis Mo.) unless otherwise noted.

### Abbreviations

Dyed Chemibeads: Carboxyl modified polystyrene latex particle comprising a chemiluminescent compound (chelated europium and thioxene) and prepared in a manner such as described in U.S. Pat. Nos. 5,811,311 and 6,406,667, the relevant disclosures of which is incorporated herein by reference.

APRM: Dyed chemibeads chemically conjugated with amine-modified dextran

B12-COOH: B12-monocarboxylate was prepared by acid hydrolysis of B12 using a procedure similar to that described by Allen, R. H., J. Biol. Chem. (1972) 247: 7695-7701.

Citrate buffer: 0.5 M potassium phosphate, 30 mg/mL hydroxypropyl cyclodextrin, 0.1% ZWITTERGENT® detergent, pH 5.0.

EDA-EPRM: Ethylenediamine modified EPRM

EDC: N-(3-Dimethylaminopropyl)-N′-ethylcarbodiimide hydrochloride

EPRM: APRM chemically conjugated with aldehyde-modified dextran.

EDTA=ethylenediaminetetraacetate

HEPES buffer: 50 mM HEPES, 300 mM NaCl, 1.0 mM EDTA, 0.15% PROCLIN® 300 preservative, 0.1 mg/mL neomycin, pH 7.20.

LOCI: luminescent oxygen channeling immunoassay (induced luminescence assay)

MES: 2-(N-morpholino)ethanesulfonic acid

MES buffer=50 mM MES, pH 6.0

MOP: 1-methoxy-2-propanol

NHS: N-hydroxysuccinimide

DI water: distilled water

PD10 column=SEPHADEX® G25 column from GE Healthcare Corporation (Waukesha, Wis.) (Part #17-0851-01)

UPA instrument=Ultra Particle Analyzer

hr=hour(s)

min=minute(s)

sec=seconds

nm=nanometer(s)

mg=milligram(s)

mL=milliliter(s)

μL=microliter(s)

rpm=revolutions per minute

cm=centimeter(s)

pg=picogram(s)

### Example 1

The EPRM chemibead is prepared in a manner similar to the method described in U.S. Pat. No. 6,153,442 and U.S. Patent Application Publication No. 20050118727A, the relevant disclosures of which are incorporated herein by reference. The EPRM chemibead comprises an aminodextran inner layer and a dextran aldehyde outer layer having free aldehyde functionalities. See, for example, U.S. Pat. Nos. 5,929,049, 7,179,660 and 7,172,906, the relevant disclosures of which are incorporated herein by reference. The reaction is carried out at a temperature of 37° C. for a period of 16 hr at a pH of 6.0, in a 50 mM MES-buffered aqueous medium. The reaction is quenched by addition of an aqueous solution of carboxymethylhydroxylamine hemihydrochloride (CMO), and the particles are subsequently washed.

Aldehyde groups on the outer dextran aldehyde layer are reacted with ethylene diamine under reductive amination conditions to form reagent EPRM-EDA having pendant moieties comprising an ethylene chain and a terminal amine group. The reductive amination conditions include the use of a reducing agent such as sodium cyanoborohydride. The reaction is carried out in an aqueous medium at a temperature during the reaction of 37° C. for a period of 16 hr.

### Preparation of EPRM-EDA Beads

EPRM beads (2000 mg, 20.0 mL) are added to a 40-mL vial. The EPRM beads are prepared by a procedure similar to that described in U.S. Pat. No. 7,179,660, the relevant disclosure is incorporated herein by reference, and the chemiluminescent compound is 2-(4-(N,N, di-tetradecyl)-anilino-3-phenyl thioxene with europium chelate. EDA (800 mg, 890 μL) is combined with 10 mL MES pH 6 buffer (the “Buffer”) and about 4.2 mL 6N HCl. The pH of the mixture is, or is adjusted to be, about 6.9. The EDA solution is added to the EPRM beads with vortexing and the mixture is rocked at room temperature for 15 min. Sodium cyanoborohydride (400 mg) is combined in a 15-mL vial with 10 mL DI water and the combination is added to the bead mixture from above. The mixture is shaken at 37° C. for 18-20 hr. The beads are transferred to six 40-mL centrifuge tubes. MES buffer is added to bring the volume to 35 mL and the mixture is centrifuged at 19,000 rpm for 30 min. The supernatant is decanted and the beads are re-suspended in 2 mL of the Buffer with a stir-rod and additional Buffer is added to 35 mL. The mixture is sonicated at 18 Watts power for 30 sec, using ice to keep the mixture cold. The wash/sonication step is performed 4 times to remove all excess unreacted EDA still present in the reaction mixture. After the last MES Buffer centrifugation, 2 mL of the Buffer containing 5% MeOP and 0.1% TWEEN® 20 surfactant (the “second Buffer”) is added to the tubes for the re-suspension step. Additional second buffer is added to 35 mL before sonication. The bead suspension is centrifuged at 19,000 rpm for 30 min. The supernatant is discarded. The final sonication used 12 mL of the second Buffer in each tube to give a 25 mg/mL dilution. Particle size is 277 nm as determined on a UPA instrument.

Preparation of Digoxin Chemibead Reagent (Coupling of Ouabain onto Detergent Pretreated EDA-EPRM)

EDA-EPRM (2 mL, 100 mg/mL particles) in 50 mM MES, pH 6.0 was mixed with 20 μL of TWEEN® 20 and mixture was shaken at room temperature for 48 hr. The mixture was passed through a PD10 column prepared and eluted with 50 mM MES buffer, pH 6.0. The treated particles were centrifuged at 15000 rpm for 30 min, supernatant solution was discarded, and solid residue was suspended in 2 mL of 50 mM MES buffer, pH 6.0.

A methanol solution of ouabain (150 mg in 3 mL methanol) (as a coupling agent/linking group) was mixed with 0.6 mL solution of periodic acid (85 mg/mL water). The reaction mixture was stirred at room temperature for 1 hr. Excess methanol was removed by flushing reaction mixture with a stream of nitrogen gas and residue was mixed with 120 μL DMF.

The detergent-pretreated EDA-EPRM was combined with 0.24 mL solution of the oxidized ouabain solution (as above) and 0.8 mL of a solution of sodium cyanoborohydride. Total mixture was shaken at 37° C. for 48 hr followed by 37° C. incubation for 16 hr after addition of a 170 μL solution of 1 M carboxymethylhydroxylamine hemihydrochloride. The ouabain-coupled particles were washed by diafiltration using a 0.1 micron 50 cm² cartridge utilizing HEPES pH 7.20 buffer; and effluents were collected after each wash cycle.

For purposes of comparison, a control coupling reaction was performed at the same time using 2 mL of EDA-EPRM and 0.24 mL of the oxidized ouabain solution exactly as described above for the detergent-pretreated EDA-EPRM. Two beads were diluted to 10 mg/mL particles using HEPES pH 7.20 buffer.

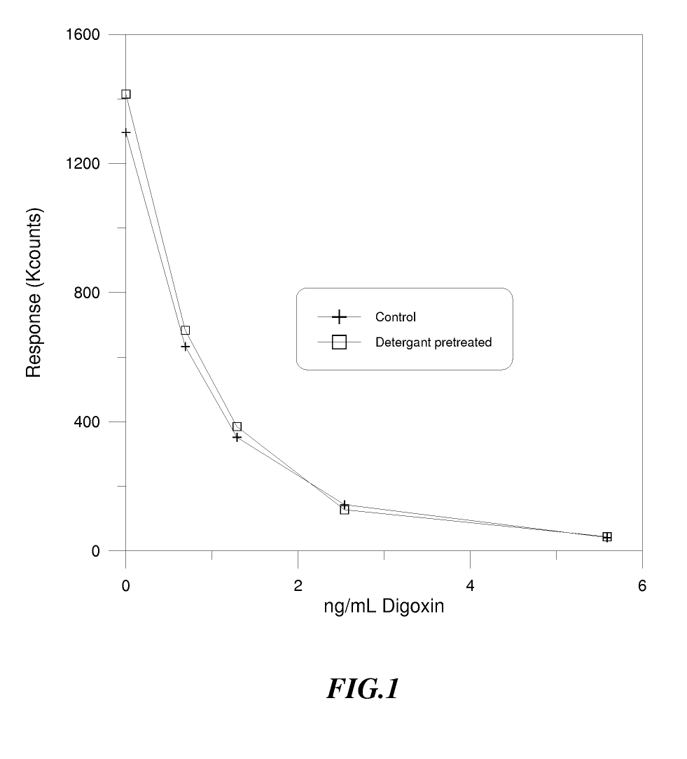

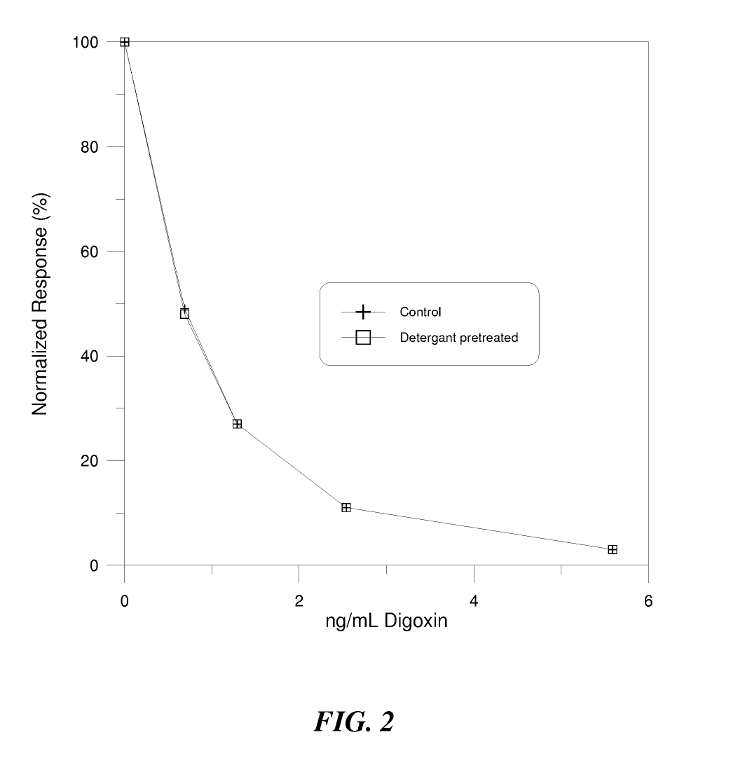

Presence of free ouabain detected by the LOCI-based digoxin assay in the supernatants during washings is shown in Table 1 below and performance of these beads is shown in FIGS. 1 and 2. Performance evaluation was carried on a DIMENSION® VISTA® analyzer (Siemens Healthcare Diagnostics Inc., Deerfield, Ill.) following the protocol for a LOCI assay using parameters used for the commercial LOCI-based digoxin assay on this platform.

TABLE 1
Ouabain content (ng/mL) in the supernatants
collected during washing of the chemibeads

| No. of wash cycles | Control | Detergent pretreated |
|---|---|---|
| 1 | 13.04 | 12.99 |
| 2 | 12.05 | 12.09 |
| 3 | 9.70 | 9.04 |
| 4 | 6.51 | 5.54 |
| 5 | 3.47 | 2.67 |
| 6 | 1.59 | 1.11 |
| 7 | 0.77 | 0.44 |
| 8 | 0.30 | 0.15 |
| 9 | 0.12 | 0.04 |
| 10 | 0.05 | 0.01 |

The results in Table 1 demonstrate that at every wash cycle free ouabain content of the supernatant of the ouabain-coupled chemibead reagent, prepared by detergent pretreatment, is lower than that of the control chemibead reagent. Thus, non-specifically adsorbed ouabain is removed using a lower number of wash cycles than would otherwise be required in cases where the chemibead reagents are prepared without such a treatment.

Dose-response curves for the chemibead reagents prepared for the digoxin assay on DIMENSION® VISTA® analyzer, with and without detergent pretreatment, are shown in FIG. 1. These curves are essentially similar suggesting little impact on the performance of the ouabain-coupled chemibead reagent on pretreatment with the detergent. To account for any differences in response at zero digoxin concentration, the response at zero digoxin concentration was considered as 100% and responses at other digoxin concentrations were normalized against this response, i.e., the responses were plotted as a percent of the response for zero digoxin concentration. These normalized response curves are shown in FIG. 2. The results again demonstrate that, even though the absolute responses may be slightly affected on detergent pretreatment especially at zero digoxin concentrations, the overall shape of the curves is not affected by detergent pretreatment in accordance with the principles described herein. Since the shapes of the curves are essentially identical, the precision and the sensitivity of the assays were not affected.

### Example 2

### Treatment of EDA-EPRM

Stock EDA-EPRM (4.8 mL: 100 mg/mL) was washed three times with a buffer containing 50 mM Na₂HPO₄-100 mM NaCl, pH 7.0, by centrifugation at 15000 rpm for 30 min and then re-suspended in the buffer. The pellet was then suspended in 5 mL of the above pH 7.0 buffer. Two 0.6 mL aliquots of this suspension were mixed separately with 2.4 mL of the above pH 7.0 buffer and 15 μL of a 40% aqueous solution of TRITON® X405 detergent in one case and 0.2 mL aqueous solution of 82 mg TWEEN® 80 detergent in the other case. Both reactions were heated at 37° C. for 23 hr and then washed four times with the above pH 7.0 buffer. The pellets were then suspended in 0.5 mL each of the above pH 7.0 buffer. Each detergent-treated EDA-EPRM suspension was mixed with 100 μL of freshly activated B12-COOH solution and 5 μL of 33% TWEEN® 20 solution. Activation of B12-COOH was carried out by a 3.5 hr reaction of a solution of B12-COOH (15.95 mg/0.55 mL of 50 mM MES buffer, pH 6.0) with 0.15 mL of an aqueous solution of NHS (200 mg/mL) and 0.3 mL of an aqueous solution of EDC (200 mg/mL). Remainder of the stock EDA-EPRM, prior to detergent treatment, was mixed with 800 μL of the active ester of B12-COOH and 254, of 33% Tween® 20. All three reactions (two utilizing detergent-pretreated EDA-EPRM and a portion of the stock EDA-EPRM) were then shaken at room temperature for 66 hr followed by heating at 37° C. for 6 hr. After centrifugation at 15000 rpm for 30 min the pellets were suspended in 0.5 M potassium acetate, 20 mg/mL dextran T40, 0.1% ZWITTERGENT®, pH 5.0; heated at 40° C. for 3 hr. After storage overnight at room temperature, the pellets were washed repeatedly (centrifugation/suspension) with the above pH 5.0 buffer. After 8 washings the suspensions were stored overnight at room temperature and then finally washed four times with HEPES buffer, pH 7.20, containing 1 mg/mL dextran T500. Supernatant solutions were saved at various washing steps and analyzed for their B12 content. Analysis was carried out on a DIMENSION® VISTA® analyzer by using these washes as samples in the commercial LOCI-based B12 assay. Results are summarized in Table 2.

TABLE 2
Presence of free B12 in supernatants (pg/mL)

| Number of washes | 8X | 8X/overnight* | Final |
|---|---|---|---|
| TRITON ® X405 | 129 | 244 | 140 |
| TWEEN ® 80 | 90 | 135 | 211 |
| Control | 202 | 292 | 438 |

*After overnight storage samples were centrifuged and supernatant obtained was tested for B12 content.

The results in Table 2 demonstrate that pretreatment of EDA-EPRM with either of the two detergents evaluated, allows for faster removal of the non-specifically adsorbed B12-COOH on the solid support. Such behavior was observed at every time point during this analysis and, most importantly, after overnight storage of the chemibead reagent (where a slow equilibrium of the nonspecifically adsorbed analyte potentially leaches back into the surrounding buffer).

A set of B12-COOH coupled EDA-EPRM before and after treatment with 0.1% TRITON® X405 was prepared as described above. In brief, 1.1 mL pH 7.0 exchanged EDA-EPRM was mixed with 1.9 ml pH 7.0 buffer and 61.2 μL of 5% TRITON® X405. After being held at 37° C. for 18 hr, the particles were exchanged with pH 7.0 buffer. The pellet suspended in 0.5 mL pH 7.0 buffer was mixed with 90 μL of freshly activated B12-COOH (9.5 mg B12-COOH/0.32 mL MES, pH 6.0, mixed with 95 μL of NHS (200 mg/mL water) and 155 μL of EDC (200 mg/mL water) and 3 μL of a 30% TWEEN® 20 solution. After reaction for 66 hr at room temperature and 3 hr at 37° C., beads were washed twice with the citrate buffer, pH 5.0. The pellet was suspended in 4 mL citrate buffer, pH 5.0, heated at 40° C. and, after overnight storage at room temperature, beads were washed with the citrate buffer, pH 5.0, and then with a buffer solution containing HEPES buffer, pH 7.20 containing 1 mg/mL dextran T500.

A control reaction was carried out in which stock EDA-EPRM exchanged in pH 7.0 buffer was reacted with the activated B12-COOH ester.

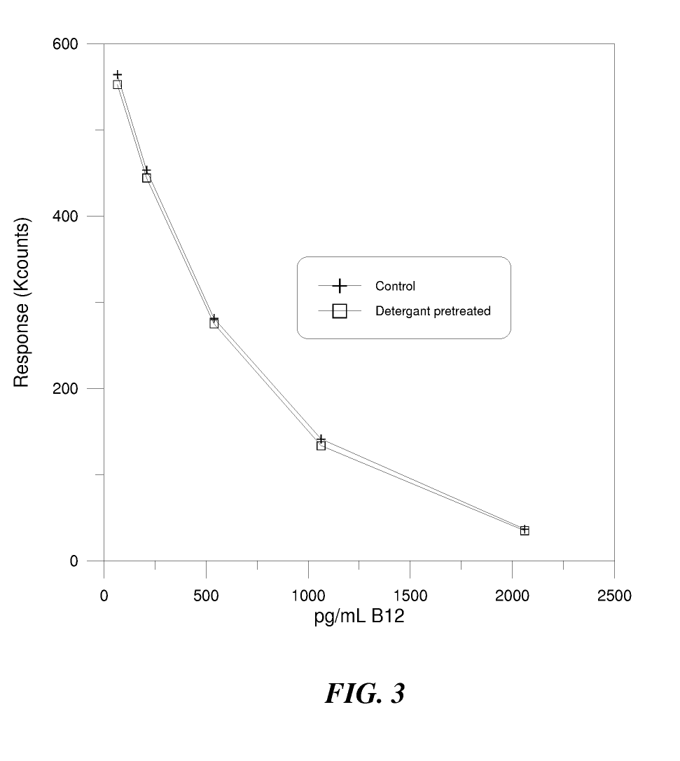

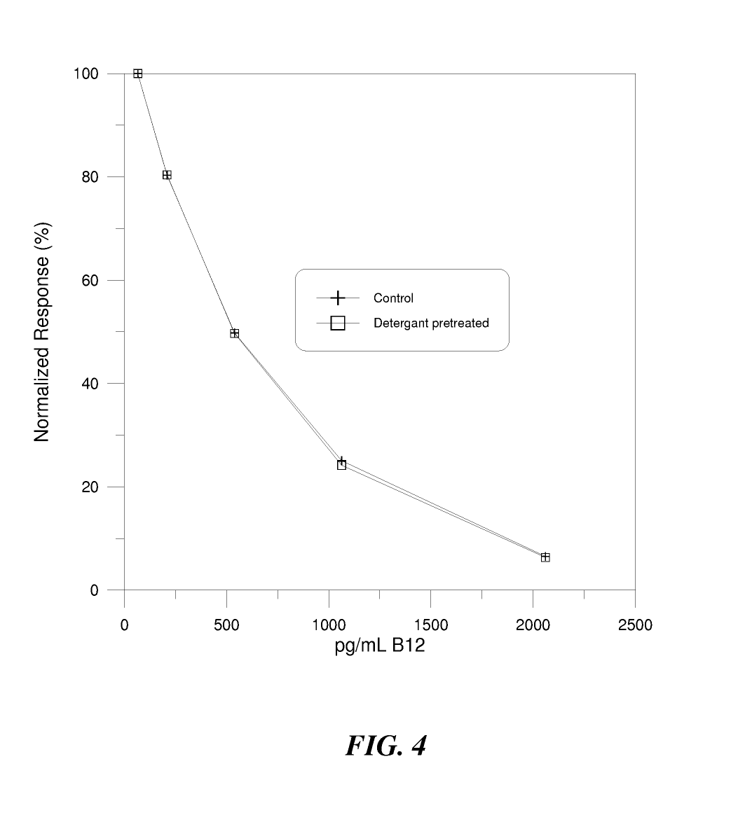

Two bead sets were each diluted to 10 mg/mL in the pH 7.2 buffer containing 1 mg/mL dextran T500 and were tested for performance on the DIMENSION® VISTA® analyzer by replacing the current commercial chemibead reagent with the reagents prepared above. Performance evaluation for the two sets of beads is shown in FIGS. 3 and 4.

Dose-response curves for the chemibead reagents prepared for the B12 assay on Dimension Vista, with and without detergent pretreatment, are shown in FIG. 3. These curves are essentially similar suggesting little impact on the performance of the B12-coupled chemibead reagent on pretreatment with the detergent. To account for any differences in response at 66 pg/mL B12, the response at 66 pg/mL B12 concentration was considered as 100% and responses at other B12 concentrations were normalized against this response, i.e., the responses were plotted as a percent of the response for the 66 pg/mL B12. These normalized response curves are shown in FIG. 4. The results in FIG. 4 demonstrate that, even though the absolute responses may be slightly affected on detergent pretreatment especially calibrators that contain 66 pg/mLB12, overall shape of the curves is not affected by this detergent pretreatment in accordance with the principles described herein. Since the shapes of the curves are essentially identical, the assay precision and the sensitivity were not affected.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method of preparing a support for reaction of the support with an assay molecule, the method comprising:
(a) treating the support with a detergent at a concentration of about 0.01% to about 5% by weight at a temperature of about 4° C. to about 50° C. for a period of about 1 hour to about 24 hours and
(b) washing the support.

2. The method according to claim 1, wherein the support is particulate.

3. The method according to claim 1, wherein the support is a latex support.

4. The method according to claim 1, wherein the detergent is a non-ionic detergent.

5. The method according to claim 1, wherein the detergent is a selected from the group consisting of polyoxyethylene detergents, polyols, and mixtures thereof.

6. A method of preparing a conjugate of a support and an assay molecule, said method comprising:
(a) treating a support that is activated for covalently binding to an assay molecule with a detergent to form a treated support, and
(b) contacting the treated support with the assay molecule under conditions for covalently binding the assay molecule to the support to form a conjugate of the support and the assay molecule.

7. The method according to claim 6, wherein prior to step (b) the treated support is washed with an aqueous medium.

8. The method according to claim 6, wherein subsequent to step (b) the conjugate is washed with an aqueous medium.

9. The method according to claim 6, wherein the support is particulate.

10. The method according to claim 6, wherein the support is a latex support.

11. The method according to claim 6, wherein the detergent is a non-ionic detergent.

12. The method according to claim 6, wherein the detergent is a selected from the group consisting of polyoxyethylene detergents, polyols, and mixtures thereof.

13. The method according to claim 6, wherein the assay molecule is selected from the group consisting of members of a specific binding pair and members of a signal-producing system.

14. The method according to claim 13, wherein the assay molecule is a member of a specific binding pair and is a small molecule.

15. The method according to claim 14, wherein the small molecule is selected from the group consisting of therapeutic drugs and their metabolites, drugs of abuse and their metabolites, vitamins and their metabolites, and hormones.

16. The method according to claim 6, wherein in step (a) the support is treated with a detergent at a concentration of about 0.01% to about 5% at a temperature of about 4° C. to about 50° C. for a period of about 1 hour to about 24 hours.

17. A method of preparing a conjugate of a particulate latex support and a small assay molecule, said method comprising:
(a) treating the particulate latex support that is activated for covalently binding to the small assay molecule with a detergent to form a treated support, and
(b) contacting the treated particulate latex support with the small assay molecule under conditions for covalently binding the small assay molecule to the particulate latex support to form a conjugate of the particulate latex support and the small assay molecule.

18. The method according to claim 17, wherein the detergent is a non-ionic detergent.

19. The method according to claim 17, wherein the detergent is a selected from the group consisting of polyoxyethylene detergents, polyols, and mixtures thereof.

20. The method according to claim 17, wherein the small molecule is selected from the group consisting of therapeutic drugs and their metabolites, drugs of abuse and their metabolites, vitamins and their metabolites, and hormones.

21. A method for determining the presence and/or amount of an analyte in a sample suspected of containing the analyte, the method comprising:
(a) providing in combination in an assay medium: the sample and reagents for conducting an assay for determining the analyte wherein one of the reagents is a particulate latex support prepared by the method according to claim 17 and wherein the small assay molecule is an analyte analog, and
(b) examining the assay medium for the presence and/or amount of a complex comprising the particulate latex support, the presence and/or amount thereof being related to the presence and/or amount of the analyte in the sample.
